(19) United States
(12) Patent Application Publication
(10) Pub. No.: US 2016/0334377 A1
(43) Pub. Date: Nov. 17, 2016

(54) SURFACE EXAMINATION OF COMPONENTS

(71) Applicant: Airbus Defence and Space GmbH, Ottobrunn (DE)

(72) Inventors: Georg Wachinger, Ottobrunn (DE); Andreas Helwig, Ottobrunn (DE); Thomas Meer, Ottobrunn (DE); Matthias Geistbeck, Ottobrunn (DE); Alois Friedberger, Ottobrunn (DE); Sebastian Heckner, Ottobrunn (DE); Johann Goebel, Ottobrunn (DE)

(21) Appl. No.: 15/152,230

(22) Filed: May 11, 2016

(30) Foreign Application Priority Data
May 11, 2015 (DE) 102015107342.0

(51) Int. Cl. G01N 30/72

(52) U.S. Cl. CPC G01N 30/7206

(57) ABSTRACT

An apparatus used for the surface examination of components. The apparatus comprises: a radiation source configured to convert at least one non-volatile substance at least partially into a gas by irradiating a sample surface of a component; and a detector unit configured to qualitatively and/or quantitatively detect the at least one substance converted into gas. A method provided for a surface examination of components. The method comprises irradiating a sample surface of a component with a radiation source configured to convert at least one non-volatile substance at least partially into a gas; and a qualitative and/or quantitative detection of the at least one substance converted into gas via a detector unit.

### CROSS-REFERENCES TO RELATED APPLICATIONS

This application claims the benefit of the German patent application No. 10 2015 107 342.0 filed on May 11, 2015, the entire disclosures of which are incorporated herein by way of reference.

### BACKGROUND OF THE INVENTION

The present invention relates to an apparatus and a method for the surface examination of components.

During the manufacture of components, in particular of elements made of fiber composite plastics, such as, for example, carbon-fiber reinforced plastic, production aids are frequently used which remain on the surface of the component after manufacture. This relates, for example, to release agents which remain adhering to the surface as residue after the demolding of the respective component.

The presence of such residues can be disadvantageous for the further processing. For example, during structural bonding, the strength of a bond is negatively influenced by surface contaminations, in particular, release agent residues. As a consequence, the processed product exhibits quality deficiencies.

In order to be able to determine whether a component is suitable for a specific processing (such as structural bonding), and in order to be able to take possible counter-measures, an examination method is therefore required, by which means it can be identified whether, whereby and to what extent a component surface is possibly contaminated.

Direct measurements of contaminations in liquid or solid form on the surface are problematical as a result of the usually very small layer thicknesses of the contaminants.

Measurement techniques which measure in the gas phase are substantially more sensitive.

DE 10 2011 102 055 A1 discloses, in particular, an apparatus for inspecting a fiber composite component for contamination. This apparatus comprises, in particular, a surface heating device and a sensor array for detecting contaminants which have been desorbed from the heated surface of the fiber composite part.

Further known, for example, is laser-induced plasma spectroscopy (for short: “LIBS”). Using this technology, the chemical element silicone can be detected in siloxane-based contaminants. A disadvantage of this technique, however is that it is not possible to distinguish between silicones and silicates with this. However, this distinction is important for the assessment of the surface in relation to subsequent bonding processes.

An analysis with the aid of a Fourier transformation infrared spectrometer (FTIR), on the other hand, is not suitable for detecting release agent residues because the concentrations are too low to be able to be measured in this way.

### SUMMARY OF THE INVENTION

It is therefore an object of the present invention to provide a technique which, in particular, enables release agent residues to be detected on fiber composite plastics and whereby the disadvantages can be eliminated.

An apparatus according to the invention is used for a surface examination of components. It comprises a radiation source which is adapted and configured to convert at least one non-volatile substance at least partially into a gas by irradiating a sample surface of a component, and a detector unit which is adapted and configured to qualitatively and/or quantitatively detect the at least one substance converted into a gas.

A method according to the invention is used for a surface examination of components. It comprises irradiating a sample surface of a component with a radiation source which is adapted and configured to convert at least one non-volatile substance at least partially into a gas. The method further comprises a qualitative and/or quantitative detection of the at least one substance converted into gas by means of a detector unit.

In this document, a substance whose evaporation number according to DIN 53170 is at least 35 is designated as “non-volatile.”

“Component” in this document is to be understood as an element which is suitable for a further processing in a manufacturing process, or that already constitutes a finished product. In particular, the term comprises pre-formed elements which can be joined together and connected to one another for the manufacture of a product, for example, in vehicle construction, in particular, in aircraft construction. Also a building material unit such as, for example, a board of a fiber composite plastic laminate is included by the term “component” in this document. Particularly preferred is an embodiment in which the component is a composite component of an aircraft or spacecraft.

The sample surface can, in each case, be a total surface of the component or a local surface section of the component; an examination of the component is made on this sample surface.

An apparatus according to the invention and a method according to the invention enable a plurality of non-volatile contaminants (in particular, high-molecular processing residues) to be measured in a gentle manner, which contaminants cannot be detected using conventional methods or only at the expense of thermal damage to the component surface. In particular, the present invention allows the detection of non-volatile substances with low vapor pressures even in low concentrations or quantities.

The radiation source can, for example comprise at least one LED unit, at least one infrared emitter and/or at least one terahertz radiation source.

Particularly advantageous is an embodiment in which the radiation source comprises at least one laser emitter (which, in particular, can be used in continuous mode and/or pulsed mode) and/or at least one plasma emitter; both variants are, in particular, suitable for performing a removing pre-treatment as mentioned further below.

According to a preferred embodiment of the present invention, the irradiation takes place during a time interval of at most 180 seconds, preferably at most 60 seconds, more preferably less than 1 second. Preferably, in this case, a temperature of at least 200° C., preferably at least 400° C., more preferably at least 500° C. is produced by the irradiation on the sample surface. Such a short-term irradiation with high energy input protects the material and at the same time is nevertheless effective in converting non-volatile substances into gas. The gas can comprise the pure non-volatile substance in its gas phase. Alternatively, or additionally, the gas can comprise a decomposition product such as, for example, a volatile combustion product (e.g., exhaust gas) which has developed as a result of the irradiation.

Corresponding to this, the detection of the at least one substance converted into gas can comprise the detection of at least one decomposition product formed during the conversion, in particular, a volatile combustion product (e.g., exhaust gas).

In particular, the irradiation can be used for a pre-treatment of the sample surface, for example, in the form of a removal of material for purification and/or activation of the sample surface. The pre-treatment can be monitored by means of the detection, according to the invention, of volatile decomposition products thereby formed. In addition, the processes of release agent removal and surface activation usually undertaken in two separate processes can be combined, which means a process-technology simplification and a saving of time.

Preferably the radiation source is adapted and configured to convert, in addition to the at least one non-volatile substance, at least one further substance, in particular, for example, at least one volatile substance (evaporation number according to DIN 53170 less than 10) into gas. In particular, the conversion of the at least one non-volatile substance can be accompanied by the conversion of at least one further (e.g., volatile or moderately volatile) substance.

Particularly preferred is an embodiment in which the detector unit comprises at least one gas chromatography ion mobility spectrometer (“GC-IMS” for short). Such a detector unit is, in particular, suitable for detecting very small quantities of substance and for detecting substance mixtures.

Preferably the apparatus according to the invention is adapted and configured to form a measurement bell around the component or adjacent to the component, which adjoins the sample surface.

According to a method according to the invention, the sample surface is preferably irradiated similarly under such a measurement bell.

The sample surface, together with the measurement bell and possibly another surface (for example a substrate), forms a boundary of the (lying around or adjacent) measurement volume. For example, the measurement volume can be pulled in an arbitrary direction onto the sample surface of the component or a substrate, or it can completely enclose the component. In particular, the prepositional expression “under a measurement bell” should not be interpreted as pointing to a vertical alignment.

The measurement volume can form an arbitrarily shaped geometrical space (such as, for example, a sphere, a hemisphere, a cylinder, a truncated cone or a rectangle, to name but a few). In particular, the term of the “measurement bell” defining the measurement volume should not be understood as a restriction of the geometry of the measurement volume.

In such embodiments comprising a measurement bell, the accuracy with which the at least one substance converted into gas is detected is improved because this substance is collected at least partially in the measurement volume and thus is distributed at least to a restricted extent in the surroundings.

In addition, such a measurement bell can prevent the escape of gas which is dangerous to health and/or malodorous which can have developed due to irradiation.

The measurement bell can comprise a seal made of a flexible material which seals the measurement volume at a transition between the measurement bell and the sample surface. Particularly when used to examine a component having a curved surface, such a seal improves an adaptation to a respective substrate so that an uncontrolled exchange of gas with the surroundings is prevented.

According to a preferred embodiment, the detector unit is located outside the measurement volume. Preferably the at least one substance converted into gas is guided or pumped from the measurement volume to the detector unit through a gas outlet located in a measurement bell and is detected there. This embodiment, in particular, has the advantage that the measurement bell can be configured to be correspondingly small and can be inserted flexibly and that nevertheless a specific analytical device (in particular, for example, a gas chromatography ion mobility spectrometer) can be used as a detector unit. In addition, the substance converted into gas can be analyzed in such a separated detector unit without the conversion process caused by the irradiation of the sample surface or the sample surface itself being adversely affected.

According a preferred embodiment of the present invention, the measurement bell is at least a part of a mobile, portable manual device. It preferably has a maximum diameter of 50 cm, preferably a maximum diameter of 3 cm, even more preferably a maximum diameter of 1 cm and/or a mass of at most 5 kg, preferably of at most 1 kg, even more preferably of at most 100 g.

Particularly preferred is an embodiment of an apparatus according to the invention which comprises a controllable gas supply line which is adapted and configured to flush the measurement volume using an experimental gas; accordingly, a method according to the invention comprises similarly a flushing of the measurement volume using experimental gas. In this way, the measurement accuracy can be increased and/or the measurement process can be controlled. In particular, ambient air, synthetic air or also an inert gas such as, for example, nitrogen or argon, are suitable as such an experimental gas.

In addition, a specific control of a quantity of gas supplied and a quantity of gas removed can prevent an incorrect air supply. Thus, in particular, the measurement accuracy can be increased even if the measurement volume is not completely sealed to the outside.

According to a preferred embodiment of an apparatus according to the invention, the radiation source is adapted and configured to irradiate the sample surface through an energy-transparent coupling-in window in the measurement bell; the energy-transparent coupling-in window thus seals the measurement volume with respect to the surroundings of the measurement bell. Similarly, according to a preferred embodiment of a method according to the invention, the irradiation is accomplished through such a coupling-in window.

The energy-transparent coupling-in window enables radiation delivered by the radiation source to enter into the measurement volume. In particular, it is preferably adapted and configured to transmit the type and intensity of the radiation used which is required for the at least partial conversion of the at least one substance into gas. This can be achieved, for example, by a material and/or a thickness which is adapted and configured to the radiation source and the (at least one) substance to be detected.

The radiation source can thus be positioned outside the measurement bell. As a result of a suitable distance of the radiation source from the sample surface, a wide scatter of the radiation can thus be achieved without the measurement volume needing to be selected to be correspondingly large which would reduce the accuracy of the detection of substance converted into gas in the measurement volume.

Advantageous is an embodiment of an apparatus, according to the invention, which comprises a process monitoring unit which is adapted and configured to detect a temperature prevailing (in particular on) at the sample surface; similarly, a method according to the invention preferably comprises such a detection of a temperature prevailing at (in particular on) the sample surface. The temperature can in this case be determined, for example, on the basis of a temperature measurement at a distance from the sample surface, for example, at a distance of at most 10 cm, preferably of at most 2 cm, even more preferably of at most 0.5 cm from the sample surface.

The radiation source and/or the detector unit can be controlled on the basis of the values measured by the process monitoring unit. In particular, a radiation intensity can be adjusted and/or varied as a function of a temperature detected by the process monitoring unit. As a result, on the one hand the conversion of the substance into gas can be optimized and on the other hand, thermal damage to the sample surface can be avoided.

An apparatus according to the invention is preferably configured at least partially as a portable manual device or as part of portable manual device. In particular, such an apparatus can easily be re-positioned at several positions on the component for a random sample examination of the component while the component itself can be left in its position. This is particularly advantageous with a large component which is difficult to move.

According to an advantageous embodiment of the method according to the invention, the sample surface is a first sample surface and the method further comprises irradiation of the second sample surface of the component using the radiation source for at least partial conversion of a non-volatile substance into a gas and qualitative and/or quantitative detection of the converted substance using the detector unit.

Preferably the method further comprises an interpolation of the values thus detected to determine a distribution of the non-volatile substance on a surface of the component.

In particular, a contaminant can thus be measured in the manner of a random sample and conclusions can be drawn regarding contamination of a component surface going beyond the examined sample surfaces. In this way, it can be determined, for example, whether the component is suitable for a structural bonding, e.g., because the contamination lies below a predetermined threshold value.

### BRIEF DESCRIPTION OF THE DRAWINGS

Preferred exemplary embodiments of the invention are explained in detail hereinafter with reference to drawings. It is understood that individual elements and components can also be combined other than shown.

In the figures schematically:

FIG. 1 shows an apparatus according to an exemplary embodiment of the present invention during a first examination; and

FIG. 2 shows the apparatus according to FIG. 1 during a second examination.

### DETAILED DESCRIPTION OF THE PREFERRED EMBODIMENTS

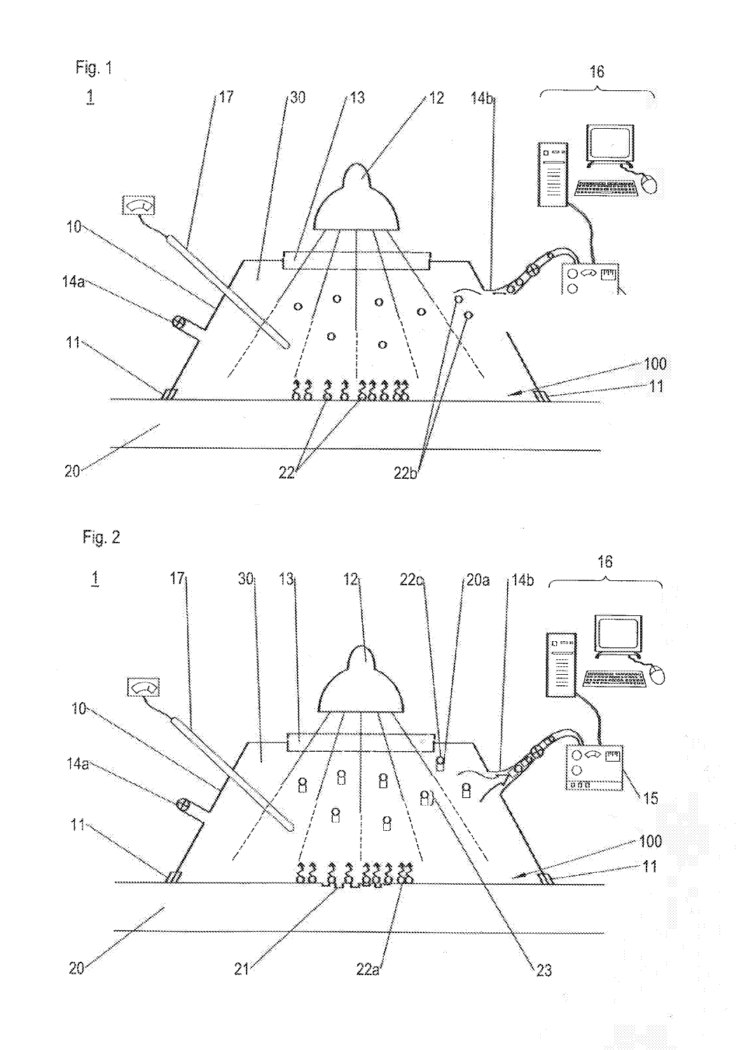

FIG. 1 shows schematically (not to scale) an arrangement comprising an embodiment of an apparatus 1 according to the invention.

The apparatus comprises a measurement bell 10 which forms a measurement volume 30 adjacent to a component 20. In particular, the measurement volume 30 is enclosed by the measurement bell and a sample surface 100 which is part of a surface of the component 20.

The measurement bell 10 has a seal 11 at the edge thereof, which seals the measurement volume at a transition between the measurement bell 10 and the sample surface 100.

In the example shown, a non-volatile substance 22a which represents a contamination in the form of a solid, viscous or liquid layer (which, for example, can comprise high-molecular release agent residues) is deposited on the component 20 which, for example, can comprise fiber-matrix composite material.

The apparatus 1 further comprises a radiation source 12 which is adapted and configured to irradiate the sample surface 100 and thereby convert the non-volatile substance 22a at least partially into gas 22b. In the exemplary embodiment shown, the radiation source is located outside the measurement bell 10 and it irradiates the sample surface through an energy-transparent window 13 located in the measurement bell 10.

A gas supply control comprises a gas supply line 14a and is adapted and configured to introduce a predetermined experimental gas (for example, ambient air or an inert gas) through the measurement volume 30 and thus control the measurement process.

In the exemplary embodiment shown, in particular, the at least one substance converted into gas is guided or pumped through a gas outlet 14b in the measurement bell to a detector unit 15 (with one or more sensors) which is adapted and configured to detect the at least one substance 22b converted into gas qualitatively and/or quantitatively; preferred is an embodiment in which the detector unit comprises at least one gas chromatography ion mobility spectrometer.

Gas supply line 14a and gas outlet 14b can additionally be used (e.g., during an interruption of the irradiation and/or detection) for flushing the measurement volume 30 and the detector unit 15 with experimental gas and/or ambient air and/or synthetic air. An accuracy of a subsequent measurement (with irradiation and detection as described) can thus be increased.

In the exemplary embodiment shown, the detector unit 15 is connected to a processor unit 16 which is adapted and configured to evaluate the values measured by the detector unit 15. In particular, the processor unit can be adapted and configured to interpolate values detected at different sample surfaces and determine a distribution of the at least one non-volatile substance on a surface comprising the sample surfaces and at least one further surface (in particular, for example, on the entire surface of the component 20).

A process monitoring unit 17 is adapted and configured to detect a temperature prevailing at the sample surface. According to a preferred embodiment, the process monitoring unit is connected to a controller of the radiation source 12 and/or to the gas supply line 14a and/or to the detector unit 15 and/or the processor unit 16 and the temperatures measured by the process monitoring unit are used for controlling the radiation source 12 or for controlling the gas supply line or for evaluating the values detected by the detector unit. In particular, the detected temperature can be compared with a predetermined threshold value which implies a risk of thermal damage to the sample surface 100. Thus, the irradiation can be terminated or the intensity of the radiation can be reduced if the temperature detected by the process monitoring unit 17 exceeds the threshold value. Alternatively, or additionally, the intensity of the radiation can be controlled in the sense of optimizing the conversion of the non-volatile substance into gas.

When the apparatus is in use, the sample surface 100 can be irradiated using the radiation source 12. As a result, the non-volatile substance 22a is converted at least partially into a gas 22b, as depicted by the arrow. The process monitoring unit 17 preferably detects the temperature prevailing at the sample surface; the radiation source can be controlled accordingly depending on the detected value, as described above.

The detector unit 15 detects the substance converted into gas, preferably several times, and relays the values detected in each case to the processor unit 16 which evaluates these values for determining any contamination of the sample surface with the non-volatile substance 22a, for example, by comparison with simulated and/or stored data.

FIG. 2 shows a similar apparatus 1 in an alternative use; the same components are provided with the same reference numbers in FIG. 1, reference being made to the above description.

In the use shown in FIG. 2, the irradiation brings about a superficial removal of the component material which is shown schematically by recesses 21 in the sample surface. The waste gas 23 formed therefore comprises decomposition products 20a of the component material in addition to the non-volatile substance 22c converted into a gas. This waste gas 23 is detected qualitatively and/or quantitatively by the detector unit 15.

Both the process shown in FIG. 1, and the use shown in FIG. 2, can each be continued over a predefined time (for example, of about 1 min. or about 10 min. or about 30 min.). Alternatively, a duration of the process can be controlled depending on values which were detected by the detector unit.

For example, the irradiation can be used as a countermeasure for the contamination (and therefore as a pre-treatment for a further process step such as, for example, a structural bonding of the component) in which the irradiation is used for the removal of the at least one non-volatile substance. In this case, the irradiation can be continued or repeated, for example, until the value detected by the detector unit for the substance converted into gas falls below a threshold value; then the process is preferably terminated. The threshold value can, for example, be selected so that it gives a tolerance limit below which contamination does not or only insignificantly adversely affects any further processing.

While at least one exemplary embodiment of the present invention(s) is disclosed herein, it should be understood that modifications, substitutions and alternatives may be apparent to one of ordinary skill in the art and can be made without departing from the scope of this disclosure. This disclosure is intended to cover any adaptations or variations of the exemplary embodiment(s). In addition, in this disclosure, the terms “comprise” or “comprising” do not exclude other elements or steps, the terms “a” or “one” do not exclude a plural number, and the term “or” means either or both. Furthermore, characteristics or steps which have been described may also be used in combination with other characteristics or steps and in any order unless the disclosure or context suggests otherwise. This disclosure hereby incorporates by reference the complete disclosure of any patent or application from which it claims benefit or priority.

### REFERENCE LIST

1 Apparatus
10 Measurement bell
11 Seal
12 Radiation source
13 Window
14a Gas supply line
14b Gas outlet
15 Detector unit
16 Process unit
20 Component
20a Decomposition product
21 Recess
22a Non-volatile substance
22b, 22c Substance converted into gas
23 Exhaust gas
30 Measurement volume
100 Sample surface

1. An apparatus for the surface examination of components comprising:
a radiation source configured to convert at least one non-volatile substance at least partially into a gas by irradiating a sample surface of a component; and
a detector unit configured to at least one of qualitatively or quantitatively detect the at least one substance converted into gas.

2. The apparatus according to claim 1, wherein the radiation source comprises at least one of an infrared emitter, a laser, a terahertz radiation source, a plasma emitter or an LED unit.

3. The apparatus according to claim 1, wherein the detector unit comprises at least one gas chromatography ion mobility spectrometer.

4. The apparatus according to claim 1, wherein the radiation source is configured to initiate a removal of material of the component from the sample surface.

5. The apparatus according to claim 1, further comprising a measurement bell configured to form a measurement volume around the component or adjacent to the component, which adjoins the sample surface.

6. The apparatus according to claim 5, further comprising a controllable gas supply line configured to flush the measurement volume with an experimental gas.

7. The apparatus according to claim 5, wherein the radiation source is configured to irradiate the sample surface through an energy-transparent coupling-in window in the measurement bell.

8. The apparatus according to claim 5, wherein the measurement bell comprises a seal made of a flexible material which seals the measurement volume at a transition between the measurement bell and the sample surface.

9. The apparatus according to claim 1, further comprising a process monitoring unit configured to detect a temperature prevailing on the sample surface.

10. The apparatus according to claim 1 comprising part of a mobile, portable manual device.

11. A method for the surface examination of components, comprising:
irradiating a sample surface of a component with a radiation source configured to convert at least one non-volatile substance at least partially into a gas; and
at least one of qualitatively or quantitatively detecting at least one substance converted into gas via a detector unit.

12. The method according to claim 11, wherein the irradiation is accomplished using at least one of an infrared emitter, a laser, a terahertz radiation source, a plasma emitter or at an LED unit comprising the radiation source.

13. The method according to claim 11, wherein the detection is accomplished using at least one gas chromatography ion mobility spectrometer comprising the detector unit.

14. The method according to claim 11, which further comprises flushing a measurement volume adjoining the sample surface with an experimental gas.

15. The method according to claim 11, wherein the irradiation is accomplished as part of a pre-treatment method for at least one of purification or activation of the sample surface, and wherein the detection of the at least one substance converted into gas comprises detection of exhaust gas which occurs during the pre-treatment method.
